(19) United States
(12) Patent Application Publication
(10) Pub. No.: US 2021/0137969 A1
(43) Pub. Date: May 13, 2021

(54) COMPOSITIONS AND METHODS FOR THE TREATMENT AND PREVENTION OF CEREBRAL ATROPHY

(71) Applicant: DUKE UNIVERISTY, Durham, NC (US)

(72) Inventors: Tatiana Segura, Durham, NC (US); Elias Sideris, Durham, NC (US)

(21) Appl. No.: 17/095,536

(22) Filed: Nov. 11, 2020

Related U.S. Application Data
(60) Provisional application No. 62/933,599, filed on Nov. 11, 2019.

(51) Int. Cl. A61K 31/728; A61P 25/00; A61K 38/08; A61K 38/10; A61K 9/16; A61K 9/00

(52) U.S. Cl. CPC A61K 31/728; A61P 25/00; A61K 9/0019; A61K 38/10; A61K 9/16; A61K 38/08

(57) ABSTRACT

The present disclosure provides biomaterial compositions and methods for the treatment of cerebral atrophy and stroke in a subject.

### CROSS REFERENCE TO RELATED APPLICATIONS

This application claims priority to U.S. Provisional Patent Application Ser. No. 62/933,599, filed Nov. 11, 2019, the contents of which is hereby incorporated by reference in its entirety.

### FEDERAL FUNDING LEGEND

This invention was made with Government support under Federal Grant No. R01NS094599 awarded by the National Institutes of Health. The Federal Government has certain rights to this invention.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 2.39 kilobytes in size, and titled 20-2001-US SequenceListing ST25.txt.

### BACKGROUND OF THE INVENTION

Stroke is the leading cause of adult long-term disability. Ischemic strokes are caused by an obstruction within a blood vessel, and account for 87% of all strokes with a 32% mortality rate. For some stroke survivors (˜90% for ischemic stroke), the injury leaves them with a serious disability. There are currently no FDA approved therapies to treat long-term disability, leaving physical therapy as their only medical treatment.

Immediately following stroke onset, the lack of oxygen and nutrients causes significant cell death, a large influx of microglia/macrophages and the activation of highly reactive astrocytes, which release pro-inflammatory cytokines and lead to further neuronal death and a clearance of cellular debris. Over time, the brain's defense mechanism is to compartmentalize the injured tissue from the surrounding tissue via an astrocytic and fibrotic scar, preventing repair in the stroke core. With time the stroke core is devoid of vessels and axons and cerebral atrophy occurs (brain shrinkage). Atrophy in the motor cortex accounts for at least a portion motor deficit in stroke patients. There are currently no therapies to prevent or treat cerebral atrophy, which is correlated with dementia, depression, and reduced motor function.

Astrocytes can both aid and obstruct stroke recovery with complete ablation of astrocytes resulting in a worse outcome after stroke. Astrocytes are able to communicate with multiple neurons via secreted and contact-mediated signals, can coordinate the development of synapses and neural circuits, yet astrocytes can limit long term repair and regeneration when a pro-inflammatory phenotype is adopted and a scar is formed.

Currently, no biomaterial has been described that can modulate the astrocyte phenotype from inflammatory to pro-regenerative. The present disclosure provides for injectable hydrogel microparticles, (e.g., “Microporous Annealed Particle” or MAP hydrogels microporous annealed particle (MAP) hydrogels) as a stroke treatment by modulation of the post-stroke astrocyte and microglia phenotypes.

### BRIEF SUMMARY OF THE DISCLOSURE

The present disclosure provides, in part, biomaterials, pharmaceutical compositions, and methods for the treatment of cerebral atrophy and stroke in a subject.

One aspect of the disclosure provides for biomaterials. In some embodiments, a biomaterial can have a polymer having a hyaluronic acid backbone, wherein the polymer can be modified with at least one peptide. In some examples, the at least one peptide can be a ligand for integrin binding. In some examples, the polymer having a hyaluronic acid backbone, can be an acytlated hyaluronic acid.

In various embodiments, the biomaterials herein can have a hydrogel. In some examples, the hydrogels herein can be a microparticle annealed porous (MAP) hydrogel. In some examples, the hydrogels herein can be hydrogel microparticles. In some examples, the biomaterials herein can be formulated in a pharmaceutical composition, which can further include a pharmaceutically acceptable carrier. In some examples, the hydrogel microparticles herein can be formulated in a pharmaceutical composition, which can further include a pharmaceutically acceptable carrier suitable for injection.

One other aspect of the disclosure provides for methods of treating or preventing cerebral atrophy in a subject. In various embodiments, the methods of treating or preventing cerebral atrophy herein can include administering to the subject a therapeutically effective amount of a biomaterial as disclosed herein into the site of a cerebral lesion. In some examples, the biomaterials herein can be administered to a subject by intravenous injection, intracerebroventricular injection, intra-cisterna magna injection, intra-parenchymal injection, or a combination thereof.

Another aspect of the disclosure provides for methods of treating a stroke in a subject. The methods of treating a stroke herein can include administering a biomaterial as disclosed to a subject, wherein the subject can be having, suspected of having, or has had at least one stroke. In some examples, the type of stroke to be treated using the methods herein can be an ischemic stroke. In some examples, methods of administering a biomaterial disclosed herein to a subject having an ischemic stroke can be performed at least until 48 hours after a diagnosis of ischemic stroke.

An aspect of the disclosure provides for kits, wherein a kit can include any biomaterial disclosed herein and at least one container.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure, which can be better understood by reference to the drawing in combination with the detailed description of specific embodiments presented herein.

FIGS. 1A-1K are schematics, images, and graphs showing the synthesis and characterization of hyaluronic acid based hydrogel microparticles (HA-HMPs) and of hyaluronic acid based microporous annealed particle (HA-MAP) hydrogel scaffolds in accordance with embodiments of the present disclosure. FIG. 1A shows a schematic of a droplet generating flow-focusing microfluidic device used to produce HMPs via a water-in-oil emulsion. FIG. 1B is a representative image of HMPs in aqueous solution allowed to fully swell after gelation and purification from oil-phase (Scale bar as shown represents 100 μm).

FIG. 1C is a graph showing a histogram of HMP sizes showing that the majority of HMPs are between 80-90 μm. FIG. 1D is a graph showing instron mechanical compression testing of MAP scaffold after annealing of HMPs which shows that the MAP scaffold exhibited bulk mechanical properties. FIG. 1E is a representative image of high molecular weight fluorescent dextran between the MAP void spaces showing interconnected porosity. FIG. 1F shows an injection schematic depicting direct injection into the stroke cavity after photothrombotic (PT) stroke. FIG. 1G is a representative image of a full section image of stroked brain treated with MAP showing MAP fills entire stroke cavity. FIGS. 11I-1K are representative serial section images of a single stroked brain treated with MAP with each progressive section ˜300 μm apart from the previous section.

FIGS. 2A-2E are images and graphs showing progression of inflammation after stroke in accordance with embodiments of the present disclosure. FIG. 2A are representative images of immunohistological staining for GFAP (glial fibrillary acidic protein), a marker for astrocytes, IBA-1 (ionized calcium binding adaptor molecule), a marker for microglial cells; and DAPI (4′,6-diamidino-2-phenylindole) a marker for nuclei in a cryosection from a single, healthy mouse brain (day 0) and a single stroked brain 5, 7, 15, 30, and 120 days after stroke.

FIG. 2B are representative images of immunohistological staining for GFAP, NF200 (Neurofilament 200), a marker for axonal processes, and DAPI in a cryosection from a single, healthy mouse brain (day 0) and a single stroked brain 5, 7, 15, 30, and 120 days after stroke.

FIG. 2C are representative images of immunohistological staining for GFAP, Glut-1 (Glucose Transporter-1), a marker for endothelial cells/vascular, and DAPI in a cryosection from a single, healthy mouse brain (day 0) and a single stroked brain 5, 7, 15, 30, and 120 days after stroke. FIG. 2D is a graph showing the quantification of the percent area of inflammation over time through GFAP and IBA-1 staining. FIG. 2E is a graph showing the quantification of the percent area of inflammation over time in axons (NF200) and vessels (Glut1).

FIGS. 3A-3I are images and graphs showing structural integrity of tissue following injection of MAP hydrogel after stroke in accordance with embodiments of the present disclosure. FIG. 3A shows a schematic of MAP scaffold directly injected in stroke cavity showing imperfect stacking of HMPs. FIG. 3B are representative images of hydrogel degradation in a single stroked brain 5, 15, 30, and 120 days after stroke. FIG. 3C are representative images of immunohistological staining for GFAP, NF200, and DAPI in a cryosection from a sham-injected, stroked brain 120 days after stroke depicting cerebral atrophy and reduction of nigrostriatal bundles in the sham condition. FIG. 3D are representative images of immunohistological staining for GFAP, NF200, and DAPI in a cryosection from a HA-MAP-injected, stroked brain 120 days after stroke depicting preservation of peri-infarct area with minimal cerebral atrophy and nigrostriatal bundles in MAP condition. FIG. 3E shows a graph depicting the quantification of cerebral atrophy comparing sham to HA-MAP treated brains. FIG. 3F shows a graph depicting the quantification of nigrostriatal bundle area comparing sham to HA-MAP treated brains. FIG. 3G are representative images of immunohistological staining for GFAP, NF200, and DAPI in a cryosection from a sham-injected, stroked brain 120 days after stroke depicting the peri-infarct and infarct space in the sham condition. FIG. 3H are representative images of immunohistological staining for GFAP, NF200, and DAPI in a cryosection from a HA-MAP-injected, stroked brain 120 days after stroke depicting the peri-infarct and infarct space in the MAP condition. FIG. 3I shows a graph depicting the quantification of axon percent area above ventricle comparing sham to HA-MAP treated brains.

FIGS. 4A and 4B graphs showing preservation of global brain size and cortex size following injection of MAP hydrogel after stroke in accordance with embodiments of the present disclosure. FIG. 4A is a graph showing the crossectional area for a sham-injected, stroked brain 120 days after stroke, a HA-MAP-injected, stroked brain 120 days after stroke wherein the HA-MAP had a ˜350 Pa storage modulus (gel 1), and a HA-MAP-injected, stroked brain 120 days after stroke, wherein the HA-MAP had a ˜800 Pa storage modulus (gel 2). FIG. 4B is a graph showing the ratio of stroke and non-stroke hemisphere area for a sham-injected, stroked brain 120 days after stroke, a HA-MAP-injected, stroked brain 120 days after stroke wherein the HA-MAP had a ˜350 Pa storage modulus (soft MAP), and a HA-MAP-injected, stroked brain 120 days after stroke, wherein the HA-MAP had a ˜800 Pa storage modulus (Stiff MAP).

FIGS. 5A-5J are schematics, images and graphs showing astrocyte and microglia function following injection of MAP hydrogel after stroke in accordance with embodiments of the present disclosure. FIG. 5A shows a schematic of the experimental timeline where arrow signifies stroke day, + signifies injection day, and x signifies sacrifice and analysis time point.

FIG. 5B shows a schematic of analysis setup showing where sections were imaged and analyzed. FIG. 5C are representative images of immunohistological staining for GFAP, DAPI, and either pERK (left panel) or S100β (right panel) in a cryosection from a sham-injected, stroked brain 120 days after stroke (top row) or a HA-MAP-injected, stroked brain 120 days after stroke (bottom row) depicting astrocyte reactivity through pERK/GFAP co-staining and S100β/GFAP co-staining. FIG. 5D is a graph showing the quantification of pERK/GFAP percent of astrocytes that are highly reactive. FIG. 5E is a graph showing the quantification of S100β/GFAP percent of astrocytes that are highly reactive. FIG. 5F are representative images of an in situ hybridization assessment of astrocyte reactivity through C3 and SLC1A2 probing in a cryosection from a sham-injected, stroked brain 120 days after stroke (right image) or a HA-MAP-injected, stroked brain 120 days after stroke (left image). FIG. 5G is a graph depicting the quantification of the percentage of C3/SLC1A2 reflecting astrocytes that were highly reactive. FIG. 5H are representative images of immunohistological staining for CD11b (cluster of differentiation molecule 11B), a marker for immune cells, DAPI, and either NOS2 (Nitric Oxide Synthase 2, also referred to as “iNOS”), a marker for pro-inflammatory microglia/macrophages (left panel) or Arginase 1 (Arg1), a marker for Pro-repair microglia/macrophages (right panel) in a cryosection from a sham-injected, stroked brain 120 days after stroke (top row) or a HA-MAP-injected, stroked brain 120 days after stroke (bottom row). FIG. 5I is a graph showing the quantification of iNOS2/DAPI for determination of microglial pro-inflammatory phenotype. FIG. 5J is a graph showing the quantification of Arg1/DAPI for determination of microglial pro-repair phenotype.

FIGS. 6A-6C are images and graphs showing microglia and CSPG deposition following injection of MAP hydrogel after stroke in accordance with embodiments of the present disclosure. FIG. 6A are representative images of immunohistological staining for Iba1 (top row), chondroitin sulfate proteoglycan (CSPG) (middle row), and both (bottom row) in a cryosection from a sham-injected, stroked brain 7 days after stroke (left) or a HA-MAP-injected, stroked brain 7 days after stroke (right). FIG. 6B is a graph showing the quantification of positive area of CSPG staining in infarct of sham- and HA-MAP-injected mouse brains 7 days after stroke. FIG. 6C is a graph showing the quantification of positive area of CSPG staining in peri-infarct of sham- and HA-MAP-injected mouse brains 7 days after stroke.

FIGS. 7A-7I are schematics, images and graphs showing astrocyte infiltration and microglia/macrophage reactivity following injection of MAP hydrogel after stroke in accordance with embodiments of the present disclosure. FIG. 7A is a schematic of experimental timeline where arrow indicates day of stroke, + indicated day of injection, and x indicates days of sacrifice and analysis. FIG. 7B are representative images of immunohistological staining for GFAP, Iba1, and DAPI in a cryosection from a healthy mouse brain (left) and a stroked brain 5 days after stroke (right) showing reactive astrocytes and microglia of healthy tissue and 5 days post stroke. FIG. 7C are representative images of immunohistological staining for GFAP (top row), Iba1 (middle row), and both (bottom row) in a cryosection from a sham-injected, stroked brain 7, 15, or 30 days after stroke. FIG. 7D are representative images of immunohistological staining for GFAP (top row, Iba1 (middle row), and both (bottom row) in a cryosection from a HA-MAP-injected, stroked brain 7, 15, or 30 days after stroke. FIG. 7E is a graph showing the quantification of scar thickness over time through GFAP staining comparing sham and HA-MAP treated brains. FIG. 7F is a graph showing the quantification of percent area of reactive astrocytes in the peri-infarct through GFAP staining comparing sham and HA-MAP treated brains. FIG. 7G is a graph showing the quantification of astrocyte infiltration into infarct through GFAP staining comparing sham and HA-MAP treated brains. FIG. 7H is a graph showing the quantification of percent area of reactive microglia in peri-infarct area through IBA-1 staining comparing sham and HA-MAP treated brains. FIG. 7I is a graph showing the quantification of percent area of reactive microglia in infarct through IBA-1 staining comparing sham and HA-MAP treated brains.

FIGS. 8A-8I are schematics, images and graphs showing axonogenesis following injection of MAP hydrogel after stroke in accordance with embodiments of the present disclosure. FIG. 8A is a schematic of experimental timeline where arrow indicated day of stroke, + indicated day of injection, and x indicates days of sacrifice and analysis. FIG. 8B are representative images of immunohistological staining for GFAP, NF200 and DAPI in a cryosection from a healthy mouse brain (left) and a stroked brain 5 days after stroke (right) showing reactive astrocytes and microglia of healthy tissue and 5 days post stroke. FIG. 8C are representative images of immunohistological staining for GFAP (top row), NF200 (middle row), and both (bottom row) in a cryosection from a sham-injected, stroked brain 7, 15, or 30 days after stroke. FIG. 8D are representative images of immunohistological staining for GFAP (top row), NF200 (middle row), and both (bottom row) in a cryosection from a HA-MAP-injected, stroked brain 7, 15, or 30 days after stroke. FIG. 8E is a graph showing the quantification of percent area of axons in the peri-infarct through NF200 staining comparing sham and HA MAP treated conditions. FIG. 8F is a graph showing the quantification of percent area of axons in the infarct through NF200 staining comparing sham and HA MAP treated conditions. FIG. 8G is a graph showing the quantification of axon infiltration distance into infarct through NF200 staining comparing sham and HA MAP treated conditions. FIG. 8H are representative images of immunohistological staining for GFAP and NF200 in a cryosection from a HA-MAP-injected, stroked brain 30 days after stroke reflecting co-residing astrocytes and axons (as magnified in the right panel). FIG. 8I are representative images of immunohistological staining for GFAP, NF200, and Sox2 in a cryosection from a HA-MAP-injected, stroked brain 120 days after stroke reflecting co-residing astrocytes and axons (as magnified in the right panel).

FIG. 9A-9F are images and graphs showing vessel density following injection of MAP hydrogel after stroke in accordance with embodiments of the present disclosure. FIG. 9A are representative images of immunohistological staining for GFAP, Glut1 and DAPI in a cryosection from a healthy mouse brain (left) and a stroked brain 5 days after stroke (right) showing reactive astrocytes and microglia of healthy tissue and 5 days post stroke. FIG. 9B are representative images of immunohistological staining for GFAP (top row), Glut1 (middle row), and both (bottom row) in a cryosection from a sham-injected, stroked brain 7, 15, or 30 days after stroke. FIG. 9C are representative images of immunohistological staining for GFAP (top row), Glut1 (middle row), and both (bottom row) in a cryosection from a HA-MAP-injected, stroked brain 7, 15, or 30 days after stroke. FIG. 9D is a graph showing the quantification of percent area of vessels in the peri-infarct through Glut1 staining comparing sham and HA MAP treated conditions. FIG. 9E is a graph showing the quantification of percent area of vessels in the infarct through Glut1 staining comparing sham and HA MAP treated conditions. FIG. 9F is a graph showing the quantification of vessels infiltration distance into infarct through Glut1 staining comparing sham and HA MAP treated conditions.

FIG. 10 is a graph showing infiltration of astrocytes (GFAP), axons (NF200), and vessels (Glut1) intro the infarct 7, 15, and 30 days after stroke in accordance with embodiments of the present disclosure.

FIGS. 11A-11F are graphs showing effects of HA-MAP stiffness on astrocyte, microglia, axon, and vessels following injection of MAP hydrogel after stroke in accordance with embodiments of the present disclosure. FIG. 11A is a graph showing the quantification of scar thickness (immunohistological staining for GFAP) in MAP-injected brains 5, 7, 15, or 30 days where the MAP-injection was either 4.5% MAP hydrogel or 3.5% MAP hydrogel. FIG. 11B is a graph showing the quantification of astrocyte infiltration (immunohistological staining for GFAP) into the infarct in MAP-injected brains 5, 7, 15, or 30 days where the MAP-injection was either 4.5% MAP hydrogel or 3.5% MAP hydrogel. FIG. 11C is a graph showing the quantification of reactive microglia (immunohistological staining for IBA-1) percent area in the peri-infarct of MAP-injected brains 5, 7, 15, or 30 days where the MAP-injection was either 4.5% MAP hydrogel or 3.5% MAP hydrogel. FIG. 11D is a graph showing the quantification of reactive microglia (immunohistological staining for IBA-1) percent area in the infarct of MAP-injected brains 5, 7, 15, or 30 days where the MAP-injection was either 4.5% MAP hydrogel or 3.5% MAP hydrogel. FIG. 11E is a graph showing the quantification of axon (immunohistological staining for NF200) infiltration into the infarct of MAP-injected brains 5, 7, 15, or 30 days where the MAP-injection was either 4.5% MAP hydrogel or 3.5% MAP hydrogel. FIG. 11F is a graph showing the quantification of vessel (immunohistological staining for Glut1) infiltration into the infarct of MAP-injected brains 5, 7, 15, or 30 days where the MAP-injection was either 4.5% MAP hydrogel or 3.5% MAP hydrogel.

FIGS. 12A-12Q are schematics, images and graphs showing cellular responses to HA-MAP formulations following injection of MAP hydrogel after stroke in accordance with embodiments of the present disclosure. FIG. 12A is a schematic of experimental timeline where arrow indicates day of stroke, + indicates day of injections, and x indicates day of sacrifice and analysis. FIG. 12B are representative images of immunohistological staining for GFAP, Iba1, and both in a cryosection from a sham-injected, stroked brain 7 days after stroke (top row) and a HA-MAP-injected, stroked brain 7 days after stroke (bottom row). FIG. 12C are representative images of immunohistological staining for GFAP, Iba1, and both in a cryosection from a non-porous HA-MAP-injected, stroked brain 7 days after stroke, where the non-porous HA-MAP hydrogel had an identical composition to HA-MAP but crosslinked as a bulk gel. FIGS. 12D-12G are graphs showing the quantification of: scar thickness (FIG. 12D); astrocyte infiltration (immunohistological staining for GFAP) into the infarct (FIG. 12E); reactive microglia (immunohistological staining for IBA-1) percent area in the peri-infarct (FIG. 12F); and, of reactive microglia (immunohistological staining for IBA-1) percent area in the infarct (FIG. 12G) of sham-injected, HA-MAP-injected, or non-porous HA-MAP-injected brains 7 days after stroke. FIG. 12H are representative images of immunohistological staining for GFAP, Iba1, and both in a cryosection from a no-RGD HA-MAP-injected, stroked brain 7 days after stroke, where the no-RGD HA-MAP hydrogel had an identical composition to HA-MAP but did not contain RGD. FIGS. 12I-12L are graphs showing the quantification of: scar thickness (FIG. 12I); astrocyte infiltration (immunohistological staining for GFAP) into the infarct (FIG. 12J); reactive microglia (immunohistological staining for IBA-1) percent area in the peri-infarct (FIG. 12K); and, of reactive microglia (immunohistological staining for IBA-1) percent area in the infarct (FIG. 12L) of sham-injected, HA-MAP-injected, or no-RGD HA-MAP-injected brains 7 days after stroke. FIG. 12M are representative images of immunohistological staining for GFAP, Iba1, and both in a cryosection from a PEG-MAP- (top row) and a no-RGD PEG-MAP- (bottom row) injected, stroked brain 7 days after stroke, where the PEG-MAP hydrogel identical composition to HA-MAP but instead of a HA backbone, a PEG (PEG-vinyl sulfone (20,000 Da)) backbone was used. FIGS. 12N-12Q are graphs showing the quantification of: scar thickness (FIG. 12N); astrocyte infiltration (immunohistological staining for GFAP) into the infarct (FIG. 12O); reactive microglia (immunohistological staining for IBA-1) percent area in the peri-infarct (FIG. 12P); and, of reactive microglia (immunohistological staining for IBA-1) percent area in the infarct (FIG. 12Q) of sham-injected, HA-MAP-injected, PEG-MAP-injected or no-RGD PEG-MAP-injected brains 7 days after stroke.

FIGS. 13A and 13B are graphs showing properties of various MAP hydrogel formulations in accordance with embodiments of the present disclosure. FIG. 13A is a graph showing microgel size distribution for 3.5% HA MAP, 4.5% HA MAP, and PEG MAP. FIG. 13B is a graph showing quantification of the Young's modulus for 3.5% HA MAP, 4.5% HA MAP, and PEG MAP.

### DETAILED DESCRIPTION

The death rate due to stroke is decreasing, resulting in more individuals living with stroke related disabilities. Following stroke, dying cells contribute to the large influx of highly reactive astrocytes and pro-inflammatory microglia that release cytokines and lead to a cytotoxic environment that causes further brain damage and prevents endogenous repair. Paradoxically, these same cells also activate pro-repair mechanisms that contribute to endogenous repair and brain plasticity.

The present disclosure is based, at least in part, on the development of a hyaluronic acid based microporous annealed particle (HA-MAP) hydrogel that, when injected into a brain lesion, promotes reparative astrocyte infiltration into the lesion. Such HA-MAP hydrogels may serve as an advantageous therapeutic option for stroke by providing neuroprotection of the infarcted area. Accordingly, described herein are hydrogels and methods of making such, pharmaceutical compositions comprising such hydrogels, and methods for delivering a hydrogel to a target site of interest, such as an infarct brain area, for treating a target disease such as brain ischemia following stroke.

### I. Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, “prevent” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human. In other embodiments, the subject comprises a human in need of bone repair or bone formation.

“Microparticles” as used herein refers to particles including polymers, having relatively small dimensions including diameter, radius, height, width, depth, etc. In embodiments, for example, microparticles refer to particles having a lateral dimension (e.g. diameter) of less than or equal to equal to 1 mm. In some embodiments, microparticles refers to particles having an average or mean diameter of less than or equal to 500 less than or equal to 100 or less than or equal to 50 In some embodiments, microparticles are microspheres. In some embodiments, microparticles refer to particles having lateral dimensions selected from the range of 10 nm to 1000 preferably for some embodiments, 10 nm to 100 μm.

“Microdroplets” can be used herein refer to microparticles in the liquid phase. For example, in some embodiments, microdroplets refer to droplets having a mean or average diameter of less than or equal to 500 less than or equal to 100 or less than or equal to 50 μm. In embodiments, microdroplets refer to liquids in a suspension, for example an emulsion. In an embodiment, microdroplets refer to aqueous liquids suspended in a non-aqueous liquid. In some embodiments, microdroplets refer to particles having lateral dimensions selected from the range of 10 nm to 1000 preferably for some embodiments, 10 nm to 100 μm.

“Hydrogel” as used herein refers to an at least partially hydrophilic substance having characterized by high water absorbency. In some embodiments, hydrogel may have an at least partially hydrophilic polymer, superabsorbent polymer or biomacromolecule, for example in a network configuration. Hydrogels may be characterized as a water swollen but insoluble substance. In embodiments, for example, hydrogels may absorb water greater than or equal to 10 times the hydrogel weight, greater than or equal to 50 times the hydrogel weight or, optionally, greater than or equal to 100 times the hydrogel weight.

Unless otherwise defined, all technical terms used herein have the same meaning as commonly understood by one of ordinary skill in the art to which this disclosure belongs.

### II. Biomaterials

In one aspect, the present invention provides for a biomaterials for the treatment of stroke. As used herein, “biomaterial” refers to any material suitable for in vivo applications. In certain instances herein, particular biomaterials of the disclosure may be referred to as hydrogels, hydrogel microparticles, or scaffolds. The biomaterials of the present invention comprise a polymer functionalized with, or conjugated to, a bioactive molecule binding moiety.

As used herein, “functionalized,” “functionalized with,” “conjugated,” and “conjugated to” are used interchangeably to refer to the chemical coupling, typically though covalent binding, of two or more molecules. Molecules may, for example, be copolymerized, or a moiety may be included as a substituent to a particular functional group or molecule. “Bioactive molecule” as used herein refers to a therapeutic agent for the treatment of diseases, disorders, and conditions, including those disclosed herein, and “bioactive molecule binding moiety” refers to a moiety able to reversibly bind to, or to dynamically covalently bind, a bioactive molecule.

As used herein, the term “hydrogel” refers to a broad class of polymeric materials, that may be natural or synthetic, have an affinity for an aqueous medium, and may absorb large amounts of the aqueous medium, but do not normally dissolve in the aqueous medium. Generally, a hydrogel can be formed by using at least one, or one or more types of hydrogel-forming agent, and setting or solidifying the one or more types of hydrogel-forming agent in an aqueous medium to form a three-dimensional network, wherein formation of the three-dimensional network may cause the one or more types of hydrogel-forming agent to gel so as to form the hydrogel. The term “hydrogel-forming agent”, also termed herein as “hydrogel precursor”, can refer to any chemical compound that may be used to make a hydrogel disclosed herein. The hydrogel-forming agent may comprise a physically cross-linkable polymer, a chemically cross-linkable polymer, or mixtures thereof. In some aspects, a hydrogel precursor can be a hydrogel microparticle. In some examples, a hydrogel microparticle for use herein can have a size ranging from about 10 μm to about 200 μm in diameter. In some examples, a hydrogel microparticle for use herein can have a size of about 10 μm, about 20 μm, about 40 μm, about 60 μm, about 80 μm, about 100 μm, about 120 μm, about 140 μm, about 160 m, about 180 μm, or about 200 μm in diameter.

In some embodiments, a hydrogel microparticle for use herein can have at least one polymeric material. In some examples, the polymeric material can be, a natural polymer material, a synthetic polymer material and combinations thereof. In some examples, a polymer suitable for use herein may be homopolymeric, heteropolymeric (including, but not limited to, cross-polymers or co-polymers of any co-monomer distribution), and may be linear, branched, hyperbranched, dendrimeric, or crosslinked to any extent. Examples of suitable polymers can include, but are not limited to, gelatin, methylcellulose, hydroxyethyl methyl cellulose, hydroxypropyl methyl cellulose, polyethylene oxide, polyacrylamides, polyacrylic acid, polymethacrylic acid, salts of polyacrylic acid, salts of polymethacrylic acid, poly(2-hydroxyethyl methacrylate), polylactic acid, polyglycolic acid, polyvinylalcohol, polyanhydrides such as poly(methacrylic) anhydride, poly(acrylic) anhydride, polysebasic anhydride, collagen, poly(hyaluronic acid), hyaluronic acid-containing polymers and copolymers, polypeptides, dextran, dextran sulfate, chitosan, chitin, agarose gels, fibrin gels, and combinations thereof. In some aspects, a polymer suitable for use herein may be a hydrophilic polymer. In some examples, a hydrophilic polymer can be selected from the group comprising poly(ethylene glycol), polyoxazoline, polyaliphatic polyurethanes, polyether polyurethanes, polyester polyurethanes, polyethylene copolymers, polyamides, polyvinyl alcohols, poly(ethylene oxide), polypropylene oxide, polypropylene glycol, polytetramethylene oxide, polyvinyl pyrrolidone, polyacrylamide, poly(hydroxy ethyl acrylate), poly(hydroxyethyl methacrylate), or mixtures or co-polymers thereof. In some examples, a hydrogel microparticle for use herein may contain the synthetic polymer poly(ethylene glycol) (PEG).

In some embodiments, a hydrogel microparticle for use herein may contain hyaluronic acid (HA). HA is a non-sulphated glycosaminoglycan (GAG) in the extracellular matrix (ECM) of many soft connective tissues, composed of alternating units of D-glucuronic acid and N-acetyl-D-glucosamine with a molecular weight (MW) up to about 6 MDa, linked together via alternating β-1,4 and β-1,3 glycosidic bonds. In some examples, HA may be extracted from natural tissues including the connective tissue of vertebrates, from the human umbilical cord and from cocks' combs. In some examples, HA may prepared by microbiological methods to minimize the potential risk of transferring infectious agents, and to increase product uniformity, quality and availability. For use herein, HA can be crosslinked to impart stability, improve function, or both. Non-limiting methods of crosslinking HA include crosslinking by bisepoxide divinyl sulfone derivatives, and the like under alkaline conditions and by glutaraldehyde, 1-ethyl-3-(3-dimethylaminopropyl) carbodiimide hydrochloride (EDC), biscarbodiimide multifunctional hydrazides, and the like under acidic conditions. In some aspects, a hydrogel microparticle for use herein may contain acrylated HA. One of skill in the art will understand how to introduce acrylate groups on HA. As an example, acrylate groups on HA can be introduced by synthesis of glycidyl methacrylate-HA (GMHA) conjugates, synthesis of N-3-aminopropyl methacrylamide-HA conjugates by modification of the carboxyl groups present in HA.

In some embodiments, a hydrogel microparticle can be formed from a hydrogel precursor solution. A “hydrogel precursor solution” as used herein can include concentrations of polymer from about 10 mg/mL to about 100 mg/mL. In some examples, a hydrogel precursor solution can include concentrations of polymer from about 10 mg/mL to about 100 mg/mL wherein the polymer is acrylated HA. In some aspects, a hydrogel precursor solution as used herein can further include at least one multifunctional polymer crosslinker. Suitable multifunctional polymer crosslinkers for use in the hydrogel precursor solution are known by those skilled in the art. A multifunctional crosslinker can be, for example, a bifunctional polymer crosslinker or a multifunctional polymer crosslinker terminated with a functional group that can form a covalent bond with the polymer of the hydrogel precursor solution. In some examples, bi-functional polymer crosslinkers and multifunctional polymer crosslinkers can be polyethylene glycol dithiol (PEG-DT), protease-degradable crosslinkers and multi-arm poly(ethylene glycol) terminated with thiol (e.g., 4-arm PEG terminated with thiol). In some other examples, suitable protease-degradable crosslinkers can be, matrix metalloproteinase (NMP)-degradable crosslinkers. Exemplary examples of suitable MMP-degradable crosslinking peptides for use in the hydrogel precursor solution herein can include

(SEQ ID NO: 1)
KCGGPQGIWGQGCK,
(SEQ ID NO: 2)
KCGGPQGIAGQGCK,

or

(SEQ ID NO: 3)
GCRDGPQGIWGQDRCG.

In some other aspects, a hydrogel precursor solution as used herein can include components needed to perform a FXIIIa-mediated crosslinking reaction. In some examples, one of at least two hydrogel precursor molecules can be functionalized with a lysine-bearing peptide sequence (K-peptide), whereas the other can be functionalized with a glutamine-bearing peptide sequence (Q-peptide). In some examples, a K-peptide can have a final hydrogel concentration of about 200 μM to about 300 μM. In some examples, a Q-peptide can have a final hydrogel concentration of about 200 μM to about 300 μM. As an example, but not limited to, a K-peptide can be Ac-FKGGERCG-NH2 (SEQ ID NO: 4) and Q-peptide can be Ac-NQEQVSPLGGERCG-NH2 (SEQ ID NO: 5). In an exemplary example, a hydrogel precursor solution as used herein can include acrylated HA, a K-peptide, and a Q-peptide.

In still some other aspects, a hydrogel precursor solution as used herein can further include a cell adhesion peptide. As used herein, a “cell adhesion peptide” refers to an amino acid sequence obtained from an adhesion protein to which cells bind via a receptor-ligand interaction. Varying the cell adhesion peptide and concentrations thereof in the solution can allow for the ability to control the stability of the cellular attachment to the resulting hydrogel composition. In some aspects, at a cell adhesion peptide can have a final hydrogel concentration of about 1 μM to about 500 μM. In some examples, a “cell adhesion peptide” can be a peptide ligand for integrin binding. Suitable peptide ligands for integrin binding include, for example, RGD, RGDS (SEQ ID NO: 6), CRGDS (SEQ ID NO: 7), CRGDSP (SEQ ID NO: 8), PHSRN (SEQ ID NO: 9), GWGGRGDSP (SEQ ID NO: 10), RGDSPGERCG (SEQ ID NO: 11). In some aspects, at a peptide ligand for integrin binding can have a final hydrogel concentration of about 1 μM to about 500 μM.

In some embodiments, a hydrogel precursor solution can be used to generate a hydrogel microparticle as disclosed herein. One of skill in the art will appreciate the methods known to generate hydrogel microparticles. Non-limiting examples of suitable methods can include bulk aqueous phase emulsification via sonication, vortexing, or homogenization, or by microfluidic drop-wise emulsification. In some exemplary examples, hydrogel microparticles can be formed by subjecting a hydrogel precursor solution as disclosed herein to a droplet generator flow focusing microfluidic device.

In some embodiments, hydrogel microparticles generated from a hydrogel precursor solution disclosed herein can be used to generate a hydrogel. In some aspects, hydrogel microparticles as described herein can be cross-linked to each to generate a hydrogel. In some aspects, hydrogel microparticles as described herein can be cross-linked to each other using the coagulation enzyme factor XIIIa (FXIIIa) to generate a hydrogel.

In some aspects, a hydrogel generated from the hydrogel microparticles disclosed herein can have a Young's modulus of about 0.5 to about 2000 Pa. In some aspects, a hydrogel generated from the hydrogel microparticles disclosed herein can have a Young's modulus of about 0.5 Pa, about 10 Pa, about 50 Pa, about 100 Pa, about 200 Pa, about 300 Pa, about 400 Pa, about 500 Pa, about 600 Pa, about 700 Pa, about 800 Pa, about 900 Pa, about 1000 Pa, about 1250 Pa, about 1500 Pa, about 1750, or about 2000 Pa.

In some aspects, a hydrogel described herein can be porous. In some examples, the pores can be homogenously dispersed throughout the hydrogel. In some other examples, the pours can be heterogeneously dispersed throughout the hydrogel. In some examples, hydrogels described herein can have micron-sized pores. In some examples, hydrogels described herein can have pores having an approximate diameter of about 0.1 μm to about 900 μm, about 1 μm to about 500 μm, or about 10 μm to about 250 μm.

Exemplary examples of a hydrogel generated from the hydrogel microparticles disclosed herein can Microporous Annealed Particle (MAP) scaffolds. MAP scaffolds can be generated from a hydrogel precursor solution disclosed herein having about 7% (w/v) to about 9% (w/v) acrylated HA. A MAP scaffold generated as described herein can have about 3% (w/v) to about 4.5% (w/v) acrylated HA. A MAP scaffold generated as described herein can have up to about 500 μM of a peptide ligand for integrin binding. A MAP scaffold generated as described herein can have a Young's modulus of about 920 Pa to about 930 Pa. A MAP scaffold generated as described herein can have pores having an approximate diameter of about 0.1 μm to about 900 μm wherein the pores can be either homogenously or heterogeneously dispersed throughout the scaffold.

### III. Pharmaceutical Compositions

Any of the biomaterials disclosed herein may be formulated to form a pharmaceutical composition, which further comprises a pharmaceutically acceptable carrier, diluent or excipient. Any of the pharmaceutical compositions to be used in the present methods can comprise pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” may refer to molecular entities and other ingredients of compositions comprising such that are physiologically tolerable and do not typically produce untoward reactions when administered to a mammal (e.g., a human). In some examples, the “pharmaceutically acceptable” carrier used in the pharmaceutical compositions disclosed herein may be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

In some embodiments, the pharmaceutical compositions or formulations are for parenteral administration, such as intravenous, intracerebroventricular injection, intra-cisterna magna injection, intra-parenchymal injection, or a combination thereof. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well known to those skilled in the art.

The pharmaceutical compositions to be used for in vivo administration should be sterile. This is readily accomplished by, for example, filtration through sterile filtration membranes. Sterile injectable solutions are generally prepared by incorporating biomaterials in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The pharmaceutical compositions disclosed herein may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycols.

### IV. Methods of Use

Any of the biomaterials (e.g., hydrogel microparticles, hydrogels, and MAP scaffolds) described herein can be used for alleviating and/or treating stroke. Thus, in some aspects, the present disclosure provides methods for alleviating one or more symptoms and/or for treating stroke in a subject in need of the treatment biomaterials disclosed herein, as well as a pharmaceutical composition comprising such.

To perform the method disclosed herein, a therapeutically effective amount of the biomaterials or a pharmaceutical composition comprising such may be administered to a subject who needs treatment via a suitable route (e.g., intravenous, intracerebroventricular injection, intra-cisterna magna injection, or intra-parenchymal injection) at a suitable amount as disclosed herein.

As used herein, the term “treating” refers to the application or administration of a composition including one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

To perform the method disclosed herein, a therapeutically effective amount of the biomaterials or a pharmaceutical composition comprising such may be administered into at least one site of a cerebral lesion. As used herein, a cerebral lesion can be any damage to an area of brain tissue caused by injury, disease, surgery, tumor, stroke, or infection. In some examples, a cerebral lesion can be any damage to an area of brain tissue caused by ischemic stroke.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

A subject to be treated by any of the methods disclosed herein may be a human patient having, suspected of having, or has previously had a stroke. Such subjects can identified by routine medical examination, e.g., laboratory tests, organ functional tests, behavioral tests, CT scans, electroencephalogram, and/or magnetic resonance imaging (MRI). In some aspects, a subject can be a human patient having, suspected of having, or has previously had an ischemic stroke, a hemorrhagic stroke, a cryptogenic stroke, a brain stem stroke, a transient ischemic attack, or a combination thereof. In some examples, a patient to be to be treated by any of the methods disclosed herein may be having, suspected of having, or has previously had an ischemic stroke. In some examples, methods disclosed should encompass administering a composition disclosed herein within at least 48 hours after a patient has been diagnosed with ischemic stroke. In some examples, methods disclosed should encompass administering a composition disclosed herein within at least 12 hours after a patient has been diagnosed with ischemic stroke.

In any of the methods disclosed herein, an effective amount of biomaterials disclosed herein can be given to a stroke patient to alleviate one or more symptoms associated with stroke. “An effective amount” as used herein refers to a dose of biomaterials, which is sufficient to confer a therapeutic effect on a subject having stroke. In some instances, symptoms associated with stroke may be behavioral, cognitive neurorehabilitation, or a combination thereof. In some examples, symptoms of stroke to be treated by methods herein can be anxiety-related and perseverative behaviors, social behaviors, learning, memory, or a combination thereof.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. Effective amounts can also vary, depending on phenotypic variability among subjects having stroke, and/or the genetic mutations involved. Such an amounts can be determined by those skilled in the art following routine practice, for example, examining blood levels of virus at multiple time points after administration to determine whether the dose is proper.

Conventional methods, known to those of ordinary skill in the art of medicine, can be used to administer the biomaterial-containing pharmaceutical composition to a stroke subject. For example, this pharmaceutical composition can also be administered parenterally, e.g., by intravenous injection, intracerebroventricular injection, intra-cisterna magna injection, intra-parenchymal injection, or a combination thereof. In some embodiments, biomaterial-containing pharmaceutical compositions can administered to the human patient via at least two administration routes. In some examples, the combination of administration routes by be intracerebroventricular injection and intravenous injection; intrathecal injection and intravenous injection; intra-cisterna magna injection and intravenous injection; and intra-parenchymal injection and intravenous injection.

In some embodiments, the subject to be treated by the method described herein may be a human patient who has undergone or is subjecting to a stroke therapy. The prior stroke therapy may be complete. Alternatively, the stroke therapy may be still ongoing. In other embodiments, the stroke patient may be subject to a combined therapy involving the biomaterial therapy disclosed herein and a second stroke therapy. Stroke therapies include, but are not limited to, IV injection of recombinant tissue plasminogen activator (tPA), endovascular therapy, clot removal by surgical intervention, carotid endarterectomy, angioplasty, surgical clipping, endovascular embolization, surgical AVM removal, stereotactic radiosurgery, or a combination thereof. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

In some embodiments, the dosage of biomaterial or a pharmacological composition thereof may be adjusted based on the patient's response to the treatment. For example, if the patient shows worsening of one or more behavior features (e.g., behavioral and/or cognitive activities), the dose of biomaterials can be reduced. Alternatively, if the patient does not show clear improvement of stroke symptoms, the dose of biomaterials may be increased.

In some embodiments, the dosage of biomaterials (e.g., hydrogel microparticles, hydrogels, MAP scaffolds) or a pharmacological compositions thereof may improve astrocyte infiltration into an area of the brain infarcted following stroke. In some aspects, the dosage of biomaterials or a pharmacological compositions thereof may increase astrocyte infiltration into an area of the brain infarcted following stroke by about 10% to about 99%.

In some embodiments, the dosage of biomaterials or a pharmacological compositions thereof may improve the scar thickness in an area of the brain infarcted following stroke. In some aspects, the dosage of biomaterials or a pharmacological compositions thereof may decrease scar thickness in area of the brain infarcted following stroke by about 10% to about 99%.

In some embodiments, the dosage of biomaterials or a pharmacological compositions thereof may reduce the number of microphage/microglia in the peri-infarct and infarct spaces following stroke in a subject. In some aspects, the dosage of biomaterials or a pharmacological compositions thereof may reduce the number of microphage/microglia in the peri-infarct and infarct spaces following stroke in a subject by about 10% to about 99%.

### V. Kits

The present disclosure also provides kits for use in treating stroke as described herein. A kit for therapeutic use as described herein may include one or more containers further including a biomaterial (e.g., hydrogel microparticles, hydrogels, or MAP scaffolds) as described herein, formulated in a pharmaceutical composition.

In some embodiments, the kit can additionally comprise instructions for use of biomaterials in any of the methods described herein. The included instructions may comprise a description of administration of the biomaterials or a pharmaceutical composition comprising such to a subject to achieve the intended activity in a subject. The kit may further comprise a description of selecting a subject suitable for treatment based on identifying whether the subject is in need of the treatment. In some embodiments, the instructions comprise a description of administering the biomaterials or the pharmaceutical composition comprising such to a subject who has or is suspected of having stroke

The instructions relating to the use of the biomaterials as described herein generally include information as to dosage, dosing schedule, and route of administration for the intended treatment. In some embodiments, the instructions comprise a description of optimizing the dose of biomaterials in a subject having stroke using one or more of the behavior features a biomarker.

The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

In some embodiments, the kit include hydrogel microparticles and FXIII for injection into the stroke cavity. In some examples, the kit can contain syringes pre-loaded with hydrogel microparticles and/or FXIII and instructions for how to administer the compositions according to methods as described herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1. Generation and Characterization of Hydrogels

Granular hydrogels for the Examples described herein were materials generated from hydrogel microparticle (HMP) building blocks using a droplet generator flow focusing microfluidic device (FIG. 1A). HMPs used in this study were ˜80 μm diameter (FIGS. 1B and 1C), generated using a hyaluronic acid (HA) backbone, and peptides as crosslinkers (MMP degradable) and ligands for integrin binding (RGD). In brief, hyaluronic acid (HA) functionalized with an acrylate was first dissolved at 7% (w/v) in 0.3 M triethyloamine (TEOA) pH 8.8 and pre-reacted with K-peptide (Ac-FKGGERCG-NH2; SEQ ID NO: 4) and Q-peptide (Ac-NQEQVSPLGGERCG-NH2; SEQ ID NO: 5) at a final hydrogel concentration of 250 μM, and RGD (Ac-RGDSPGERCG-NH2; SEQ ID NO: 11) at a final hydrogel concentration of 500 μM. Concurrently, the cross-linker solution was prepared by dissolving the di-thiol matrix metalloproteinase (MMP) sensitive peptide (Ac-GCRDGPQGIWGQDRCG-NH2; SEQ ID NO: 3) in distilled water at 7.8 mM and reacted with 10 μM Alexa-Fluor 647-maleimide for 5 minutes. These solutions were mixed in a flow focusing microfluidic device and then immediately pinched by 1% span-80 in heavy mineral oil to form microspheres. These microspheres were collected and allowed to gel overnight at room temperature to form microgels. The microgels were then purified by repeated washes with HEPES buffer (pH 7.4 containing 10 mM CaCl₂)) and centrifugation.

The 4.5% microgels used in the Examples herein were produced in the same manner, however, the HA-acrylate precursor solution was dissolved at 9% (w/v). The HA without RGD microgels used in the Examples herein were produced with the same precursor solution as the 3.5% HA, however, no RGD was added. The PEG microgels used in the Examples herein were produced in a similar manner where 4-arm PEG-Vinyl sulfone was dissolved at 10% (w/v) and the peptide concentrations were the same as the HA solutions.

Nanoporous hydrogel precursor solutions were exactly the same as the microgel precursor solutions. Additionally, the same enzyme sensitive di-thiol cross-linker solution was prepared. These two solutions were thoroughly mixed in an Eppendorf tube by vortexing and pipetting. Five U/mL of FXIII and 1 U/mL of Thrombin were added to the solution and the nanoporous hydrogel was allowed to gel in situ via the same Michael type addition in which the microgels were individually formed.

HMPs were cross-linked to each other using the coagulation enzyme factor XIIIa (FXIIIa) to generate a stable scaffold, with a Young's Modulus of ˜927 Pa (FIG. 1D). In brief, the microgels were pelleted by centrifuging at 18,000 G and the supernatant was discarded to form a concentrated solution of microgels. Five U/mL of FXIII and 1 U/mL of Thrombin were combined in the presence of 10 mM Ca′ with the pelleted μgels and allowed to incubate at 37° C. for 90 minutes between two glass slides (1 mm thickness) surface coated with a solution of a chlorinated organopolysiloxane in heptane that readily forms a covalent, microscopically thin film on glass). The mechanical testing on the hydrogel scaffolds was done using a 5500 series Instron. After annealing, the scaffolds were allowed to swell in HEPES buffer saline for 4 hours at room temperature (27° C.±5° C.). A 2.5 N load cell with a 3.12 mm tip in diameter was used at a compression strain rate of 1 mm/min and the hydrogel scaffold was indented 0.8 mm or 80% of its total thickness.

The resulting linked HMP hydrogels were termed Microporous Annealed Particle (MAP) scaffolds. FIG. 1E shows an image of high molecular weight fluorescent dextran between the MAP-void spaces showing interconnected porosity. Also, the MAP scaffolds had micron sized voids (pores) formed in between packed beads (FIG. 1E).

### Example 2. Use of MAP Scaffolds in an In Vivo Photothrombotic Stroke Model

To model ischemic stroke, Examples described herein used a photothrombotic (PT) stroke model. This model allowed examination of the brain tissue's response at short term (5-15 days post stroke) and long-term time points. In brief, a cortical photothrombotic stroke was induced on 8-12 week old, male C57BL/6 mice. The mice were anesthetized with 2.5% isoflurane and placed onto a stereotactic setup. The mice were kept at 2.5% isoflurane in N₂O:O₂ for the duration of the surgery. A midline incision was made and Rose Bengal (4,5,6,7-tetrachloro-2′,4′,5′,7′-tetraiodafluorescein) (10 mg/mL) was injected intraperitoneally into the mouse at 10 μL/g of mouse body weight. After 5 minutes of Rose Bengal injection, a 2-mm diameter cold fiberoptic light source was centered at 0 mm anterior/1.5 mm lateral left of the bregma for 18 minutes and a burr hole was drilled through the skull in the same location. All mice were given sulfamethoxazole and trimethoprim oral suspension (TMS (303 mL TMS/250 mL H₂O)) every 5 days for the entire length of the experiment.

Five days following stroke surgery, microgels with FXIII were loaded into a Hamilton syringe connected to a pump and 6 μL of microgels were injected into the stroke cavity using a 30-gauge needle at stereotaxic coordinates 0.26 mm anterior/posterior (AP), 3 mm medial/lateral (ML), and 1 mm dorsal/ventral (DV) with an infusion speed of 1 μL/min (FIG. 1F). The needle was withdrawn from the mouse brain 5 minutes after the injection to allow for microgel annealing. For each condition a minimum of 5 mice was used. HA-MAP scaffolds injected into the stroke core at 5-days post wounding did not deform the recipient hemisphere (FIG. 1G). HA-MAP completely filled the stroke core shown in serial sections where each progressive section was ˜300 μm apart (FIGS. 1H-1K).

To assess the inflammatory response the in photothrombotic stroke model over time, mice were sacrificed via transcardial perfusion of 0.1 M PBS followed by 40 mL of 4 (w/v) % PFA seven days, fifteen days, thirty days, and one hundred twenty days following stroke as induced as described in Example 2 herein. After sacrifice, the brains were isolated and post-fixed in 4% PFA overnight and submerged in 30 (w/v) % sucrose solution for 24 hours. Tangential cortical sections of 30 μm-thickness were sliced using a cryostat and directly mounted on gelatin-subbed glass slides for immunohistological staining of GFAP (glial fibrillary acidic protein), a marker for astrocytes; IBA-1 (ionized calcium binding adaptor molecule), a marker for microglial cells; Glut-1 (Glucose Transporter-1), a marker for endothelial cells; NF200 (Neurofilament 200), a marker for axonal processes; pERK (phosphorylated extracellular signal-regulated kinase), a marker for highly reactive astrocytes; S100β (calcium-binding protein β), a marker for highly reactive astrocytes; CD11b (cluster of differentiation molecule 11B), a marker for immune cells; Arginase 1, a marker for Pro-repair microglia/macrophages; NOS2 ((Nitric Oxide Synthase 2), a marker for pro-inflammatory microglia/macrophages; Sox2 (SRY-Box Transcription Factor 2), a marker for neural progenitor cells; and DAPI (4′,6-diamidino-2-phenylindole) a marker for nuclei. Primary antibodies (1:100) or DAPI (1:500) were incubated overnight at 4° C. and secondary antibodies (1:1000) were incubated at room temperature for two hours. A Nikon C2 confocal microscope was used to take fluorescent images. The IBA-1, GFAP, pERK, 510013, CD11b, Arg1, iNOS, NF200, and Glut-1 astrocytic (GFAP) and positive area in the infarct and peri-infarct areas were quantified in 4 to 8 randomly chosen regions of interest (ROI of 0.3 mm²) at a maximum distance of 300 μm from the infarct for the peri-infarct analysis. In each ROI, the positive area was measured.

Time course analysis of macrophage/microglia (IBA1+ cells) and astrocytes (GFAP+ cells) in the infarct and peri-infarct spaces revealed an early peak (7-days) of IBA1+ cells in the core of the infarct, which subsequently subsides reaching a significantly lower level by 30-days (FIGS. 2A-2C). The peri-infarct percent area of microglia/macrophages cells and astrocytes increase by 7-days and subsequently plateau through 30-days (FIG. 2D). These data mirror the expected dogma of an early inflammatory response that subsides over time. Analyses of vessels and axons in the peri-infarct space reveal that the vessel area remains relatively constant over time, while the axonal area significantly decreases over time reaching a plateau at 7-days post stroke (FIG. 2E). These data show that in the absence of any treatment, little recovery of the peri-infarct axons and vessels is found in this model.

### Example 3. Injection of MAP Hydrogel Preserved Long Term Structural Integrity and Function

In order to understand the feasibility of using HA-MAP hydrogel for brain repair, it was first investigated how long the gel lasts in the brain after implantation and if they are any detrimental effects of hydrogel injection over time (FIG. 3A). HA-MAP hydrogel was injected 5-days after stroke using the methods disclosed in Example 2 and hydrogel degradation was assessed by monitoring hydrogel fluorescence at in cryosections of brains of mice harvested days 2, 10, 30, 120 days post injection (FIG. 3B). As shown in FIG. 3B, HA-MAP hydrogel did not degrade significantly until 120-days post implantation with a significantly decreased fluorescence indicating that the HA-MAP scaffolds provided long lasting mechanical support to the tissue.

The brain tissue was subjected to immunohistological staining of cryosections of brain harvested from mice at the longest time point ˜120-days post injection. In brief, cryofrozen sections of the harvested brain tissue were allowed to thaw at room temperature. Sections were washed with PBS for 5 minutes with 3 repetitive washes. Sections were incubated with a 10% donkey serum and PBS with 0.3% triton at room temperature for one hour. The liquid was wicked away and primary antibodies of GFAP (glial fibrillary acidic protein) for astrocytes and NF200 (Neurofilament 200) for axonal processes at 1:100 dilution in PBS with 0.3% triton and 10% donkey serum were added and incubated overnight at 4° C. The next day, the primary antibodies were washed with 3 repeated PBS washes of 5 minutes each. Secondary antibodies with donkey hosts along with DAPI at 1:1000 dilution in PBS with 0.3% triton and 10% donkey serum were added and incubated at room temperature for 2 hours. After two hours, the secondary antibodies were washed away with 3 repeated PBS washes of 5 minutes each. The sections were allowed to dry at room temperature and mounted using DPX mounting medium. Analyses were performed on microscope images of 3 coronal brain levels at +0.80 mm, −0.80 mm and −1.20 mm according to bregma, which consistently contained the cortical infarct area. Large scale 10× images of each section was taken and analyzed for ventricular hypertrophy. The ratio of the ipsilateral length from the top of the section to the top of the ventricle was divided by the ratio of the contralateral length from the top of the section to the top of the ventricle was taken to get a quantitative number for the ventricular hypertrophy. Large scale 20× images were taken by the side of ventricle to analyze for nigrostriatal bundle area. Using pixel threshold on 8-bit converted images using ImageJ (Image J v1.43) and expressed as the area fraction of positive signal per area (%). Values were then averaged across all areas and sections, and expressed as the average positive area per animal. The percent area positive for NF200 was analyzed 0-1 mm out from the ventricle. Immunohistological staining of cryosections of brain harvested from mice at 120-days post injection showed long-lasting mechanical support was accompanied by maintenance of brain shape over time, which was significantly different from sham-treated mouse brain (FIGS. 3C and 3D). In particular, the scar tissue in the sham group imposed considerable fibrotic response to the brain tissue resulting in cerebral atrophy, whereby the ventricle was pulled upward toward the cortex effectively shrinking the cortex (FIG. 3E). In contrast, mice treated with HA-MAP hydrogel 5-days post stroke effectively decreased this response, which significantly prevented the deformation of the stroke cavity. In human patients, cerebral atrophy in the motor cortex accounts for at least a portion of the worsening motor deficit in stroke patients over time. The density of axons at 120-days post stroke was assessed between sham and HA-MAP treated groups at the area between the top of the ventricle to the surface of the cortex. The axonal area (NF200+ staining) was significantly higher for the HA-MAP treated mice compared to sham, further supporting our findings of reduced cerebral atrophy, which is associated with decreased neuronal cell area (FIG. 3I).

Atrophy can be observed in stroke patients and is known to cause effects at regions far away from the stroke core. Thus, cryosections of brain harvested from mice at the longest time point˜120-days post injection—were assessed to determine if there were other visible effects of a long-lasting hydrogel in the brain post stroke and reduced cerebral atrophy in mice. The striatal white matter bundles, which include the nigrostriatal bundles, were visible with NF200 staining, and were more preserved in the brains treated with HA-MAP hydrogel compared to sham brains (FIG. 3F). Quantification of the percent positive area of NF200 stain in the bundle area revealed that brains treated with HA-MAP hydrogel were more than double the percent area of the sham condition. Nigrostriatal bundles are a dopaminergic pathway that travels from the substantia nigra to the striatum and loss of these bundles can be associated with decreased motor function and is commonly observed in Parkinson's patients. Parkinson patients also observe significant cerebral atrophy, associated with worsening disease state. Further, quantification of the percent positive area of NF200 stain in the area directly above the ventricle showed a significant increase in axon area in the HA-MAP treated brains compared to the sham (FIGS. 3G-3I). Taken together the reduced cerebral atrophy and thus, preservation of these bundles suggested that the MAP gel would better preserve the motor function associated with these bundles.

To determine if the composition of the MAP gel affects global brain size or cortex size, MAP hydrogels were prepared where one gel had a ˜350 Pa storage modulus and a second MAP hydrogel had a ˜800 Pa storage modulus. The hydrogels were injected individually into mice 5-days after stroke using the methods disclosed in Example 2. Brains of mice were then harvested days 2, 10, 30, 120 days post injection and the brain crossectional area was measured. FIG. 4A shows that the brain in the sham treated groups (no hydrogel injection post stroke) decreased by 120 days where as the gel treated groups had no statistical change in crossectional area indicating that the size and shape of the brain is maintained. FIG. 4B shows the ratio of stroke and non-stroke hemisphere area for sham and gel treated groups. A ratio of 1 indicated that both hemispheres were the same size. Brains in sham treated groups decreased by 120 days. Gel treated groups had no statistical change in crossectional area except between 5 and 14-days in Gel 1 (˜350 Pa storage modulus treatment group), indicating that the size and shape of the brain is maintained in the gel treated conditions. Gel 2 (˜800 Pa storage modulus) had no statistically significant change for any of the time points. Taken together, data showed that the global brain size and cortex size changed more in the sham rodent stroke model than did that of the MAP gel-injected rodent stroke model.

### Example 4. Highly Reactive, Scar Forming, Astrocytes were Reduced after HA-MAP Injection

The use of HA-MAP hydrogels to modulate astrocyte phenotype was assessed. HA-MAP hydrogel was injected 5-days post stroke and tissue was collected 2-days post injection according to methods disclosed in the Examples herein to assess astrocyte and microglial reactivity (FIGS. 5A and 5B).

To assess astrocyte reactivity by immunohistochemisty (IHC), cryofrozen sections of harvested brain tissue were allowed to thaw at room temperature. Sections were washed with PBS for 5 minutes with 3 repetitive washes. Sections were incubated with a 10% donkey serum and PBS with 0.3% triton at room temperature for one hour. The liquid was wicked away and primary antibodies of GFAP (glial fibrillary acidic protein) for astrocytes and pERK for highly reactive astrocytes at 1:100 dilution in PBS with 0.3% triton and 10% donkey serum were added and incubated overnight at 4° C. The next day the primary antibodies were washed with 3 repeated PBS washes of 5 minutes each. Secondary antibodies with donkey hosts along with DAPI at 1:1000 dilution in PBS with 0.3% triton and 10% donkey serum were added and incubated at room temperature for 2 hours. After two hours, the secondary antibodies were washed away with 3 repeated PBS washes of 5 minutes each. The sections were allowed to dry at room temperature and mounted using DPX mounting medium. Analyses were performed on microscope images of 3 coronal brain levels at +0.80 mm, −0.80 mm and −1.20 mm according to bregma, which consistently contained the cortical infarct area. Each image represents a maximum intensity projection of 10 to 12 Z-stacks, 1 μm apart, captured at a 20× magnification with a Nikon C2 confocal microscope using the NIS Element software. For the sham sections, using ImageJ and converting to 8-bit, a ratio of positive pERK area divided by positive GFAP area within the same area was taken to get percent of reactive astrocytes that are highly reactive in the peri-infarct area 0-300 μm from the infarct border. For the HA MAP sections, the peri-infarct were analyzed similarly to sham. The infarct was analyzed by taking 0-100 μm infiltration into the lesion. S100β was stained, imaged, and analyzed similarly to pERK.

FIG. 5C shows that in the peri-infarct area there was a significantly higher percentage of reactive astrocytes in the sham condition expressing pERK1/2 (˜56% compared to −25% in MAP), an astrocytic downstream pathway for high reactivity. Moreover, significantly fewer infiltrating astrocytes express pERK1/2 were observed compared to those in the peri-infarct (˜12% in the infarct compared to −25% in the peri-infarct) (FIG. 5D). This data suggested that the astrocytes that were infiltrating the MAP gel were less reactive and potentially more pro-reparative. FIG. 5E shows a significantly higher percentage of reactive astrocytes expressing S100β in the sham condition (˜69%) compared to the 3.5% MAP gel (˜26%), with high expression of S100β considered pathological. Similar to pERK expression, the infiltrating astrocytes expressing S100β (˜15%) were significantly less that in the peri-infarct area (˜26%).

Additionally, quantitative RNA fluorescence in situ hybridization (RNA-FISH) was used to dive deeper into the reactive astrocyte phenotype. In brief, brain tissue sections were stored at −80° C. and equilibrated to room temperature before use. The brain tissue-mounted slides were immersed in 4% paraformaldehyde fixation buffer for 10 minutes at room temperature. Tissue was then permeabilized with 70% (v/v) ethanol for 24 hours at 4° C. Ethanol was aspirated and wash buffer was added. After incubation for 5 minutes at room temperature, the coverglass was transferred face-down onto Parafilm with 100 μL of hybridization Buffer containing hybridization probes for C3 (a marker shown to be upregulated in neurotoxic reactive astrocytes) and SLC1A2 (a marker for all astrocytes). After incubation for 16 hours in the dark at 37° C. in a sealed humidified chamber, the coverglass was washed with Wash Buffer A in the dark at 37° C. for 30 minutes. Nuclei were counterstained with Hoechst 33342 for 30 minutes, the tissue was then washed and prepared for visualization. Analyses were performed on microscope images of 3 coronal brain levels at +0.80 mm, −0.80 mm and −1.20 mm according to bregma, which consistently contained the cortical infarct area. Large scale 40× images were taken using a Nikon C2 confocal along the stroke border. The images were then analyzed for C3 and SLC1A2 in a manner similar to that used for positive pERK and S100β staining as detailed above.

Using a ratio of C3/SLC1A2 to analyze what percentage of astrocytes are highly reactive, it was observed that 54.2% of all astrocytes were reactive in the sham condition, while only 25.2% of all astrocytes were reactive in the 3.5% MAP gel peri-infarct condition. (FIGS. 5F and 5G). Comparing the peri-infarct of the MAP gel (˜25.2%) to the infarct of the MAP gel (˜10.7) showed that the infiltrating astrocytes are less reactive and suggests a pro-regenerative phenotype (FIG. 5G). All these results indicated that the MAP gel was able to attenuate astrocyte reactivity just two days after injection by reducing the number of highly reactive neurotoxic astrocytes by greater than 2-fold, promoting a less pro-inflammatory environment in the peri-infarct area and stimulating pro-recovery astrocyte infiltration in the infarct. Since inflammatory responses occur early after injury and initial inflammatory responses have long lasting effects in the tissue, early modulation of the astrogliotic response can have long lasting effects.

Microglial polarization directly affects astrocyte reactivity, with microglia that exhibit the more reactive M1 phenotype influencing astrocytes to a more highly reactive state. HA-MAP hydrogel was injected 5-days post stroke and brain tissue was harvested 2-days later in a manner similar to that described in the Examples above. Cryofrozen sections of the harvested brain tissue were allowed to thaw at room temperature before sections were washed with PBS for 5 minutes with 3 repetitive washes. Sections were incubated with a 10% donkey serum and PBS with 0.3% triton at room temperature for one hour. The liquid was wicked away and primary antibodies of CD11b for immune cells, Arginase 1 for Pro-repair microglia/macrophages, NOS2 for pro-inflammatory microglia/macrophages at 1:100 dilution in PBS with 0.3% triton and 10% donkey serum were added and incubated overnight at 4° C. The next day the primary antibodies were washed with 3 repeated PBS washes of 5 minutes each. Secondary antibodies with donkey hosts along with Dapi at 1:1000 dilution in PBS with 0.3% triton and 10% donkey serum were added and incubated at room temperature for 2 hours. After two hours, the secondary antibodies were washed away with 3 repeated PBS washes of 5 minutes each. The sections were allowed to dry at room temperature and mounted using DPX mounting medium. Analyses were performed on microscope images of 3 coronal brain levels at +0.80 mm, −0.80 mm and −1.20 mm according to bregma, which consistently contained the cortical infarct area. Each image represents a maximum intensity projection of 10 to 12 Z-stacks, 1 μm apart, captured at a 20× magnification with a Nikon C2 confocal microscope using the NIS Element software. In the peri-infarct for both sham and HA MAP, the ratio of the positive area of iNOS or Arg1 was divided by the positive area for CD11b from 0-300 μm from the infarct border. In the infarct, the ratio of the positive area of iNOS or Arg1 was divided by the positive area for CD11b from 0-300 μm from the infarct border.

Pro-repair microglia/macrophages were defined as expressing Arg1while pro-inflammatory microglia/macrophages expressing iNOS phenotype (FIG. 511). Interestingly, the percentage of microglia/macrophages that expressed the M1 pro-inflammatory phenotype was similar across both conditions in the peri-infarct and in the infarct (FIG. 5I). Further, the percentage of microglia/macrophages that expressed the M2 pro-repair phenotype was similar across both conditions in the peri-infarct area. However, the percentage of pro-repair microglia/macrophages in the infarct, where the cells were encapsulated within HA-MAP during injection, was almost 3-fold higher in the MAP gel condition compared to sham condition (FIG. 5J). This suggested that the infarct in the MAP gel has a more pro-reparative environment than the infarct in the sham condition. Confinement of macrophages has been shown to prevent LPS polarized M1 macrophages to activate late stage inflammatory genes. These data combined with findings of increased percentage of Arg1+ cells, suggested that microporous hydrogels formed using ˜80 μm HMPs have pore sizes that could spatially confine the microglia and lower M1 polarization.

Chondroitin sulfate proteoglycans (CSPGs) have been linked to decreased regenerative potential in the CNS. HA-MAP hydrogel was injected 5-days post stroke and brain tissue was harvested 2-days later in a manner similar to that described in the Examples above. Immunohistochemistry was performed in a manner similar to that described in the Examples above where the primary, probing antibody was and anti-CSPG. Images were assessed in a similar manner as described above.

The amount of CSPGs in the HA-MAP treated mice significantly decreases in both the infarct (˜5.3%) and peri-infarct (˜9.8%) compared to sham infarct (˜59.7%) and peri-infarct (˜38%) (FIGS. 6A-6C). Several studies that have delivered Chondroitinase ABC to digest the high release of CSPG after brain injury showed doing so had behavioral functional benefits. Thus, a material such as HA-MAP that can decrease the CSPG level without additional enzyme delivery can be advantageous.

### Example 5. Astrocytes Continued to Infiltrate Stroke Cavity Lesion Over Time

One key difference between injection of HA-MAP into the stroke cavity compared to any other hydrogels that had been tested was that HA-MAP elicited astrocyte infiltration into the stroke cavity. Thus, rather than astrocytes forming a scar around the stroke core, as occurs in sham conditions, astrocytes changed their morphology and begin to infiltrate. Data presented herein with the PT stroke model showed that this finding was not model or cortex location specific.

To investigate if this change in morphology affected the scar thickness and if astrocyte infiltration was continuous over time, HA-MAP was injected in mice 5-days after stroke using methods similar to those described in the Examples herein. Brain tissue was harvested from the mice either 7, 15, or 30-days post stroke (FIG. 7A). Cryosections of the harvested brain tissues were assessed for scar thickness and astrocyte infiltration over time by quantifying the thickness of the GFAP+ dense layer surrounding the stroke and infiltration distance of GFAP+ cells starting from the stroke border using an IHC method similar to that described in the Examples above (FIG. 7B). Sham animals (those subjected to stroke but not injected with HA-MAP) had scar thicknesses and peri-infarct % GFAP+ area that increased over time (7-30 days after stroke) (FIG. 7C), indicative of a developing scar. In contrast, injection of HA-MAP hydrogel into the stroke cavity significantly reduced the scar thickness and peri-infarct % GFAP+ area within 2-days of implantation and remained low throughout (FIGS. 7D-F).

Astrocyte (GFAP+) cell infiltration was examined over time using an IHC method similar to that described in the Examples above. It was observed that astrocyte infiltration began within 2-days of hydrogel injection and continued to increase from 7-30-days post stroke, reaching close to 500 μm into the lesion by day 30 (FIG. 7G). The infiltration pathway closely mimicked what was the expected void structure of the hydrogel (FIG. 1E), indicating that the cells infiltrated through the void space rather than through the HMPs. This infiltration pattern further suggested that significant gel degradation was not required for astrocyte infiltration and that any degraded extracellular matrix (ECM) or new ECM deposition within the void space of HA-MAP did not prevent infiltration of astrocytes. Such promotion of infiltration of pro-regenerative astrocytes into the infarct can be beneficial towards recovery. This data also showed that the MAP gel sustained astrocyte infiltration over time without the need for biologic delivery such as growth factors or small molecules.

Next, microglia/macrophage reactivity over time was determined using a IHC method similar to those used in the Examples above, staining for IBA-1 (FIG. 7D). At two days post injection, a 3-fold or 6-fold decrease in microglia stain for the peri-infarct (FIG. 7H) and infarct (FIG. 7I) area was observed, respectively, when comparing sham to HA-MA-treated strokes. Reactive microglia have been shown to contribute to highly reactive astrocytes. Further examination into microglial progression showed a very elevated total number of reactive microglia even 30 days post stroke (FIGS. 7H and 7I). Remarkably, the total number of reactive microglia at 30 days post stroke in the infarct of the sham condition was still 3-fold higher than the infarct of the HA-MAP condition at 7 days post stroke, the peak of inflammation.

### Example 6. Astrocytes and Axons Co-Infiltrated the Stroke Core after HA-MAP Injection

Given the significant impact that HA-MAP had on the phenotype of astrocytes and the number of microglia, it was next assessed if changes in the stroke environment were accompanied with increases in axonogenesis. HA-MAP hydrogel was injected into the stroke cavity 5-days after stroke and axonal area (NF200+) in the peri-infarct as well as infiltration distance in the infarct quantified using IHC methods described in the Examples above (FIGS. 8A and 8B).

The peri-infarct percent NF200+ area decreased from 53.2% to 20.4% comparing un-injured brain and stroke brain at 5-days, indicating the rapid loss of neurofilaments in the peri-infarct space after stroke (FIG. 8C). The stroke cavity was devoid of neurofilaments at this timepoint. Axons (NF200+) increased in the peri-infarct space following injection of HA-MAP hydrogel, with a significant increase at 7-days post stroke compared with sham (FIGS. 8D-8F). However, this increase was non-significant at the 15- and 30-day timepoints. This data suggested that the lower number of reactive astrocytes combined with the decreased number in macrophage/microglia in the HA-MAP condition, promoted a pro-reparative environment early after HA-MAP injection that promoted axonal sprouting, but this environment could not be maintained. However, our earlier data at 120-days showed that as cerebral atrophy occurs, axons are maintained in the HA-MAP condition but reduced in sham (FIGS. 3C-3H).

In the infarct cavity, axonal infiltration for the HA-MAP hydrogel treated groups was observed, but not in the sham groups. The path of infiltration was similar to what the astrocytes follow, navigating between the HMPs rather than through the HMPs (FIG. 8G). The infiltration distance of axons steadily increased over time reaching ˜450 μm by day 30 (FIG. 8G). Staining for both astrocytes (GFAP) and axons (NF200) revealed that not only did the axons follow the same path, but the axons appeared to localize closely with the infiltrating astrocytes (FIGS. 8H and 8I). High magnification image analysis revealed that although GFAP staining existed without the axonal stain, the reverse was not true—whenever an axonal stain was present it was closely associated with GFAP staining. Examining the HA-MAP hydrogel condition at 4-month revealed that the close association of axons and NF200 was maintained and that it was also accompanied by progenitor cells (SOX2+) (FIG. 8I). Although these images could not determine the ratio of GFAP+, NF200+, and GFAP+/SOX2+ cells, all three cell types were likely present within HA-MAP hydrogel at 4 months post injection. Both recruitment of endogenous NPCs and transplantation of exogenous NPCs have been used as strategies to promote stroke repair and shown increase behavioral response. This astrocyte/axon correlation suggested that the astrocytes were crucial for the axon penetration and maintenance, indicating that promotion of pro-repair astrocyte infiltration can be beneficial for downstream tissue repair. P astrocytes, in very near proximity to axons can be crucial in forming synapses and neuronal circuits directly in the lesion site where such recovery was not normally observed after stroke. Having astrocytes, axons and NPCs in this same space can be further manipulated to generate the desired circuitry. These results demonstrated that injection of HA-MAP hydrogel into the stroke cavity generated a permissible environment in the peri-infarct and infarct spaces that led to axonal infiltration into the stroke core.

### Example 7. Vessel Infiltration Did not Coincide with Astrocyte/Axonal Infiltration

MAP hydrogels were injected individually into mice 5-days after stroke using the methods disclosed in the Examples above. Brains of mice were then harvested days 7, 15, and 30 days post injection and cryosections prepared. Using IHC methods similar to those described in the Examples herein, an initial increase in vessel density (Glut-1+) in the peri-infarct space was observed to be statistically significant at the 15-day time-point compared to sham mice (FIGS. 9A-9C). This result was consistent with other observations of stroke-induced angiogenesis in the peri-infarct space. However, by 30-days post stroke, there was no observed statistical significance in vessel density between HA-MAP hydrogel injection and sham (FIGS. 9A-9C). Thus, similar to the axonal sprouting data, the increase in angiogenesis suggested that the lower microphage/microglia number and reduced reactive astrocytes in the peri-infarct space generated an environment that further promoted angiogenesis early after HA-MAP gel injection, but that cannot be maintained long term.

After observing significant astrocyte/axonal infiltration into the stroke core, it was assessed if vessels also infiltrate the stroke core and if they follow a similar infiltration pattern (FIGS. 9D-9F). It was observed that vessels infiltrated the stroke core, reaching a statistically significant difference of infiltrating vessels between brains treated with HA-MAP and sham groups at 30-days post injection. The infiltration path was similar to that of astrocytes and axons, following the void space between HMPs, but there appeared to be no correlation between astrocyte infiltration and vessel infiltration, unlike what was observed for axonal infiltration. The vessel stain Glut-1 did not coincide with the astrocyte stain GFAP, indicating that vessels invaded the stroke core independently. Further, it was found that the vessel infiltration distance was significantly lower compared to what was found for astrocytes and axons (FIG. 10). The average infiltration distance at 30-days for vessels was ˜207 μm while it was ˜467 μm for axons and astrocytes. This difference in infiltration distance suggests that HA-MAP can promote the infiltration of astrocytes and axons but not vessels. A few possibilities for the reduced infiltration distance for vessels are the physical properties of the gel, mechanical properties, and porosity. It is difficult to know what the mechanical properties of the gel are over time; however, the initial HA-MAP gel Young's modulus was 1000 Pa. The porosity of the scaffold is another possibility; HA-MAP gels with HMPs of 80-100 μm in diameter have a median pore area of ˜200 μm′ which would correspond to 15-20 μm when measured per z-stack. Regardless of the reason, it has been demonstrated that a robust vascular network within the stroke, promoted the formation of a neurovascular niche that led to effective axonogenesis and behavioral improvement. Thus, introducing pro-angiogenic cues into HA-MAP can promote vascularization. An additional rationale for further promoting vascularization is the fact that astrocytes and axons stopped infiltrating 200 μm away from vessels, which is the oxygen diffusion limit. Thus, lack of vascular infiltration could limit the range of astrocyte and axonal infiltration.

### Example 8. Moderate Hydrogel Stiffness Changes Did not Affect Astrocyte Behavior

Biomaterial and substrate stiffness has been shown to regulate astrocyte reactivity with softer substrates promoting astrocyte quiescence, suggesting that softer substrates should be used to better modulate astrocytes following stroke. Stiffness has also been implicated in axonal sprouting, vessel sprouting and microglia polarization.

A moderately stiffer HA-MAP hydrogel (3.5%=1000 Pa, 4.5%=1500 Pa) was generated to determine if gel stiffness change can affect astrocyte behavior. All other parameters, RGD concentration, HMP diameter, and void fraction were kept constant. MAP hydrogels were injected individually into mice 5-days after stroke using the methods disclosed in the Examples above. Brains of mice were then harvested days 7, 15, and 30 days post injection and cryosections prepared. Using IHC methods similar to those described in the Examples herein, scar thickness (GFAP), astrocyte infiltration (GFAP) into the infarct, reactive microglia (IBA-1) percent area in the peri-infarct and infarct, axon (NF200) infiltration into the infarct, and vessel (Glut1) infiltration into the infarct were assessed.

Overall, the 4.5% MAP hydrogel produced very similar results compared to the 3.5% hydrogel for astrocyte infiltration and scar thickness (FIGS. 11A and 11B). However, observed was an increased number of reactive microglia in the infarct and in the peri-infarct at 15 days post stroke, but no significant differences were observed at 30 days post stroke (FIGS. 11C and 11D). Interestingly, these increased microglia response may have caused some downstream a slight decrease in axon penetration was decreased compared to the 3.5% condition (FIGS. 11E and 11F). It is possible that the increased number of microglia in the 4.5% condition created a more cytotoxic environment that affected the axon penetration.

### Example 9. Porosity, Hyaluronic Acid, and RGD were Crucial for Astrocyte Infiltration

HA-MAP were produced from ˜80 μm HMPs, which contained HA (70,000 Da, 3.5%), RGD (500 μM), K and Q peptides (250 μM), and 7.8 mM of MMP crosslinker. The MAP gel was crosslinked using FXIIIa (5 U/mL) and 1 U/mL of thrombin. MAP hydrogels were injected individually into mice 5-days after stroke using the methods disclosed in the Examples above. Brains of mice were then harvested days 7 days post injection and cryosections prepared. Using IHC methods similar to those described in the Examples herein, scar thickness (GFAP), astrocyte infiltration (GFAP) into the infarct, and reactive microglia (IBA-1) percent area in the peri-infarct and infarct were assessed.

To test the role of microstructure on the observed findings, the observed results from HA-MAP were compared to those of a hydrogel with identical composition but crosslinked as a bulk gel (non-porous) (FIGS. 12A-12G). As a comparison we tested the ability of these gels to promote astrocyte infiltration, reduce scar thickness and reduce the number of microphage/microglia in the peri-infarct and infarct spaces. It was found that the scar thickness in the non-porous group was significantly reduced compared to sham but significantly larger compared to HA-MAP (FIG. 12D). There was also no infiltration of astrocytes into the stroke cavity (FIG. 12E), suggesting that the microstructure of MAP can be critical to this process. Microglia/macrophage area was significantly decreased in the infarct but not the peri-infarct compared to sham (FIGS. 12F and 12G); however, the decrease was not as great as that observed with HA-MAP.

Next, the effect of the integrin binding ligand RGD was tested (FIGS. 12H-12L). Injection of HA-MAP (-RGD) revealed that the scar thickness was again lower than sham, but not as low as HA-MAP (FIG. 12I). Similar to HA non-porous there was no infiltration of astrocytes into the stroke cavity, pointing to the importance of RGD in this process (FIG. 12J). The number of reactive microglia for HA-MAP(-RGD) was similar to HA-MAP for the peri-infarct space (FIG. 12K), but significantly higher for infarct (FIG. 12L). Overall, it was observed that RGD had a major effect in the ability of HA-MAP to modulate astrocyte and microglia phenotype.

Last, the role of the hydrogel backbone was tested and compared HA to polyethylene glycol (PEG) (FIGS. 12M-12Q). PEG-vinyl sulfone (20,000 Da) was used to generate HMPs using the same concentration of K, Q and MMP peptides as described in the above Examples. PEG HMPs were generated with RGD or no RGD. PEG HMPs had the same diameter as HA HMPs (FIG. 13A) and the storage modulus was the same as HA-MAP (FIG. 13B). Gels having a PEG backbone in the HMPs significantly reduced the effect observed with HA-MAP. Scar thickness was significantly thinner than sham, but larger than HA-MAP and there is no significant difference between PEG-MAP and PEG-MAP(-RGD) (FIG. 12N). Astrocyte infiltration in the PEG-MAP or PEG-MAP (-RGD) conditions was also close to zero (FIG. 12O). Finally, the microphage/microglia area was significantly decreased in the infarct for both PEG-MAP or PEG-MAP(-RGD) (FIG. 12Q), but not statistically different from sham in the peri-infarct space (FIG. 12P).

Taken together, this data showed that HA-MAP composition and microstructure was essential for the observed astrocyte infiltration, reduced scar thickness, and reduced microphage/microglia in the peri-infarct and infarct spaces. No other condition tested was equally effective as HA-MAP. Importantly, all the conditions that used HA as the backbone significantly decreased the scar thickness compared to conditions that used PEG as the backbone, demonstrating that the biological activity of HA played an important role in this process. After stroke, CD44 expression was elevated in reactive astrocytes. Thus, receptor mediated astrocyte/HA binding can be responsible for the infiltration in HA-MAP but not PEG-MAP. Implanting porous HA-RGD hydrogels into a cortex showed that RGD increased astrocyte infiltration into the cortex wound. These results agree with the finding that HA-MAP but not HA-MAP(-RGD) promoted astrocyte infiltration. It was surprising that PEG-MAP and HA-MAP resulted in different tissue responses. These materials had the same microstructures and same concentration of RGD. Given the large number of studies conducted using PEG hydrogels for tissue repair applications, it is possible that the results observed here were particular to brain and not generalizable to all tissues.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise.

The present disclosure shall control in the event there are any disparities between any definitions and/or description found in the cited references.

We claim:

1. A biomaterial comprising a polymer having a hyaluronic acid backbone, wherein the polymer is modified with at least one peptide.

2. The biomaterial of claim 1, wherein the at least one peptide comprises a ligand for integrin binding.

3. The biomaterial of claim 1, wherein the polymer having a hyaluronic acid backbone, comprises acytlated hyaluronic acid.

4. The biomaterial of claim 1, wherein the biomaterial comprises a hydrogel.

5. The biomaterial of claim 4, wherein the hydrogel comprises a microparticle annealed porous hydrogel (MAP).

6. The biomaterial of claim 1, wherein the biomaterial comprises hydrogel microparticles.

7. The biomaterial of claim 1, wherein the biomaterial is formulated in a pharmaceutical composition, which further comprises a pharmaceutically acceptable carrier.

8. The biomaterial of claim 1, wherein the biomaterial comprises hydrogel microparticles formulated in a pharmaceutical composition, which further comprises a pharmaceutically acceptable carrier suitable for injection.

9. A method of treating or preventing cerebral atrophy in a subject, the method comprising administering to the subject a therapeutically effective amount of a biomaterial into the site of a cerebral lesion, wherein the biomaterial comprises a polymer having a hyaluronic acid backbone, wherein the polymer is modified with at least one peptide.

10. The method of claim 9, wherein the at least one peptide comprises a ligand for integrin binding.

11. The method of claim 9, wherein the polymer having a hyaluronic acid backbone, comprises acytlated hyaluronic acid.

12. The method of claim 9, wherein the biomaterial comprises a hydrogel.

13. The method of claim 12, wherein the hydrogel comprises a microparticle annealed porous hydrogel (MAP).

14. The method of claim 9, wherein the biomaterial comprises hydrogel microparticles.

15. The method of claim 9, wherein the biomaterial is formulated in a pharmaceutical composition, which further comprises a pharmaceutically acceptable carrier.

16. The method of claim 9, wherein the biomaterial comprises hydrogel microparticles formulated in a pharmaceutical composition, which further comprises a pharmaceutically acceptable carrier suitable for injection.

17. The method of claim 9, wherein the biomaterial is administered to the subject by intracerebroventricular injection, intra-cisterna magna injection, intra-parenchymal injection, or a combination thereof.

18. A method of treating a stroke in a subject, the method comprising administering the biomaterial of claim 7 to a subject, wherein the subject is having, suspected of having, or has had at least one stroke.

19. The method of claim 18, wherein the stroke comprises an ischemic stroke.

20. The method of claim 18, wherein the administering of the biomaterial to a subject in need thereof occurs at least until 48 hours after a diagnosis of ischemic stroke.

21. The method of claim 18, wherein the biomaterial is administered to the subject by intracerebroventricular injection, intra-cisterna magna injection, intra-parenchymal injection, or a combination thereof.

22. A kit comprising the biomaterial of claim 1, further comprising at least one container.


SEQUENCE LISTING (11 sequences)

SEQ ID NO: 1 - protein, 14 aa, Artificial Sequence
  synthesized sequence
KCGGPQGIWGQGCK

SEQ ID NO: 2 - protein, 14 aa, Artificial Sequence
  synthesized sequence
KCGGPQGIAGQGCK

SEQ ID NO: 3 - protein, 16 aa, Artificial Sequence
  synthesized sequence
GCRDGPQGIWGQDRCG

SEQ ID NO: 4 - protein, 8 aa, Artificial Sequence
  synthesized sequence
FKGGERCG

SEQ ID NO: 5 - protein, 14 aa, Artificial Sequence
  synthesized sequence
NQEQVSPLGGERCG

SEQ ID NO: 6 - protein, 4 aa, Artificial Sequence
  synthesized sequence
RGDS

SEQ ID NO: 7 - protein, 5 aa, Artificial Sequence
  synthesized sequence
CRGDS

SEQ ID NO: 8 - protein, 6 aa, Artificial Sequence
  synthesized sequence
CRGDSP

SEQ ID NO: 9 - protein, 5 aa, Artificial Sequence
  synthesized sequence
PHSRN

SEQ ID NO: 10 - protein, 9 aa, Artificial Sequence
  synthesized sequence
GWGGRGDSP

SEQ ID NO: 11 - protein, 10 aa, Artificial Sequence
  synthesized sequence
RGDSPGERCG